(19) United States
(12) Patent Application Publication
(10) Pub. No.: US 2020/0056137 A1
(43) Pub. Date: Feb. 20, 2020

(54) CELL CULTURE SUBSTRATE COMPRISING A COPOLYMER AND A STRUCTURAL UNIT

(71) Applicant: TERUMO KABUSHIKI KAISHA, TOKYO (JP)

(72) Inventors: Takao ANZAI, KANAGAWA (JP); Ichiro HIRAHARA, KANAGAWA (JP)

(73) Assignee: TERUMO KABUSHIKI KAISHA, TOKYO (JP)

(21) Appl. No.: 16/541,848

(22) Filed: Aug. 15, 2019

(30) Foreign Application Priority Data
Aug. 16, 2018 (JP) 2018-153268

(51) Int. Cl. C12M 1/12; C12M 1/00; C12N 5/00

(52) U.S. Cl. CPC C12M 25/14; C12M 25/02; C12N 2533/30; C12N 5/0068; C12M 23/20

(57) ABSTRACT

The invention is to provide a cell culture substrate excellent in cellular adhesiveness regardless of a shape or design thereof. Provided is a cell culture substrate comprising a coating layer on at least one side of a polymer substrate, wherein the coating layer includes at least one member selected from the group consisting of a copolymer (a) comprising 40% by mole or more and 90% by mole or less of a structural unit (a-1) derived from alkoxyalkyl (meth)acrylate of following Formula (1) and 10% by mole or more and 60% by mole or less of a structural unit (a-2) derived from trialkyl aminoalkyl (meth)acrylate of following Formula (2), a copolymer (b) comprising 85% by mole or more and 95% by mole or less of a structural unit (b-1) derived from alkoxyalkyl (meth)acrylate of following Formula (1) and 5% by mole or more and 15% by mole or less of a structural unit (b-2) derived from carboxyalkyl (meth)acrylate of following Formula (3), and a copolymer (c) comprising 70% by mole or more and 90% by mole or less of a structural unit (c-1) derived from alkoxyalkyl (meth)acrylate of following Formula (1) and 10% by mole or more and 30% by mole or less of a structural unit (c-2) derived from hydroxyalkyl (meth)acrylate of following Formula (4).

### TECHNICAL FIELD

The present invention relates to a cell culture substrate excellent in cellular adhesiveness, and a bioreactor and a method for culturing a stem cell using the cell culture substrate.

### BACKGROUND

In recent years, a cell culture technology has been used in the development of regenerative medicine or drug discovery. In particular, attention has been paid to use of stem cells, and technology for repairing and replacing damaged or defective tissues has been actively studied by using stem cells expanded from donor cells. Most of cells of animals including humans are adherent (scaffold-dependent) cells which cannot survive in a floating state and survive in a state of being adhered to something. For this reason, various developments of functional culture substrates for culturing adherent (scaffold-dependent) cells at high density to obtain cultured tissues similar to living tissues have been conducted.

As a cell culture substrate, plastic (for example, polystyrene) or glass vessels have been conventionally used, and it has been reported that a plasma treatment or the like is performed to a surface of these cell vessels (for example, Patent Literature 1). Patent Literature 1 discloses that a substrate subjected to the treatment has excellent adhesion to cells, and can be used to grow cells and maintain their function.

### CITATION LIST

### Patent Literatures

Patent Literature 1: JP 63-198977 A

### SUMMARY OF THE INVENTION

The cell culture substrate subjected to the plasma treatment is certainly excellent in cellular adhesiveness. Meanwhile, regarding a structure of the cell culture substrate (cell culture vessel), in addition to a conventional flat dish (plate) structure, various structures, such as a structure in which a porous body is inserted as a culture scaffold in a bag, a hollow fiber structure, a sponge structure, a flocculent (glass wool) structure, and a structure in which a plurality of dishes are laminated, have been developed. It is difficult or impossible to perform plasma irradiation to culture vessels having such diversified and complicated structures.

Therefore, the present invention has been made in view of the above-described circumstances, and an object thereof is to provide a cell culture substrate (cell culture vessel) having excellent cell adhesion regardless of a shape or design of a cell culture substrate (cell culture vessel).

The present inventors have conducted intensive studies to solve the above-described problems. As a result, the present inventors have found that the problems can be solved by coating a surface of a cell culture substrate (polymer substrate) with an alkoxyalkyl (meth)acrylate-trialkyl aminoalkyl (meth)acrylate copolymer, an alkoxyalkyl (meth)acrylate-carboxyalkyl (meth)acrylate copolymer, or an alkoxyalkyl (meth)acrylate-hydroxyalkyl (meth)acrylate copolymer having a specific composition and structure. The present invention has been completed on the basis of the above finding.

That is, the object can be achieved by a cell culture substrate (substrate for cell culture) comprising a coating layer on at least one side of a polymer substrate, wherein the coating layer contains at least one member selected from the group consisting of

(a) a copolymer (a) comprising 40% by mole or more and 90% by mole or less of a structural unit (a-1) derived from alkoxyalkyl (meth)acrylate of following Formula (1) and 10% by mole or more and 60% by mole or less of a structural unit (a-2) derived from trialkyl aminoalkyl (meth)acrylate of following Formula (2) (a total of the structural unit (a-1) and the structural unit (a-2) is 100% by mole),

(b) a copolymer (b) comprising 85% by mole or more and 95% by mole or less of a structural unit (b-1) derived from alkoxyalkyl (meth)acrylate of following Formula (1) and 5% by mole or more and 15% by mole or less of a structural unit (b-2) derived from carboxyalkyl (meth)acrylate of following Formula (3) (a total of the structural unit (b-1) and the structural unit (b-2) is 100% by mole), and

(c) a copolymer (c) comprising 70% by mole or more and 90% by mole or less of a structural unit (c-1) derived from alkoxyalkyl (meth)acrylate of following Formula (1) and 10% by mole or more and 30% by mole or less of a structural unit (c-2) derived from hydroxyalkyl (meth)acrylate of following Formula (4) (a total of the structural unit (c-1) and the structural unit (c-2) is 100% by mole).

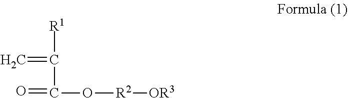

wherein R¹ represents a hydrogen atom or a methyl group, R² represents an alkylene group having 2 or 3 carbon atoms, and R³ represents an alkyl group having 1 to 3 carbon atoms,

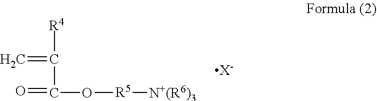

wherein R⁴ represents a hydrogen atom or a methyl group, R⁵ represents an alkylene group having 2 or 3 carbon atoms, R⁶ each independently represents an alkyl group having 1 to 3 carbon atoms, and X⁻ represents an anion moiety,

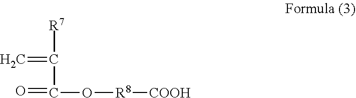

wherein R⁷ represents a hydrogen atom or a methyl group, and R⁸ represents an alkylene group having 2 or 3 carbon atoms,

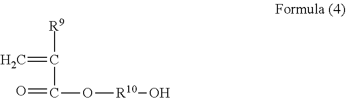

wherein R⁹ represents a hydrogen atom or a methyl group, and R¹⁰ represents an alkylene group having 2 or 3 carbon atoms.

### BRIEF DESCRIPTION OF DRAWINGS

FIG. 1 is a partial side view illustrating an embodiment of a bioreactor (hollow fiber type bioreactor) of the present invention.

FIG. 2 is a partially cut-away side view of the bioreactor of FIG. 1.

### DETAILED DESCRIPTION

The cell culture substrate (substrate for cell culture) of the present invention has a coating layer on at least one side of a polymer substrate, wherein the coating layer includes at least one member selected from the group consisting of

(a) a copolymer (a) comprising 40% by mole or more and 90% by mole or less of a structural unit (a-1) derived from alkoxyalkyl (meth)acrylate of following Formula (1) and 10% by mole or more and 60% by mole or less of a structural unit (a-2) derived from trialkyl aminoalkyl (meth)acrylate of following Formula (2) (a total of the structural unit (a-1) and the structural unit (a-2) is 100% by mole),

(b) a copolymer (b) comprising 85% by mole or more and 95% by mole or less of a structural unit (b-1) derived from alkoxyalkyl (meth)acrylate of following Formula (1) and 5% by mole or more and 15% by mole or less of a structural unit (b-2) derived from carboxyalkyl (meth)acrylate of following Formula (3) (a total of the structural unit (b-1) and the structural unit (b-2) is 100% by mole), and

(c) a copolymer (c) comprising 70% by mole or more and 90% by mole or less of a structural unit (c-1) derived from alkoxyalkyl (meth)acrylate of following Formula (1) and 10% by mole or more and 30% by mole or less of a structural unit (c-2) derived from hydroxyalkyl (meth)acrylate of following Formula (4) (a total of the structural unit (c-1) and the structural unit (c-2) is 100% by mole).

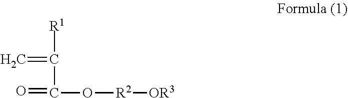

wherein R¹ represents a hydrogen atom or a methyl group, R² represents an alkylene group having 2 or 3 carbon atoms, and R³ represents an alkyl group having 1 to 3 carbon atoms.

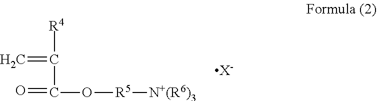

wherein R⁴ represents a hydrogen atom or a methyl group, R⁵ represents an alkylene group having 2 or 3 carbon atoms, R⁶ each independently represents an alkyl group having 1 to 3 carbon atoms, and X⁻ represents an anion moiety.

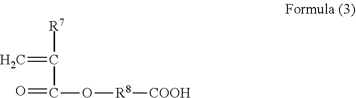

wherein R⁷ represents a hydrogen atom or a methyl group, and R⁸ represents an alkylene group having 2 or 3 carbon atoms.

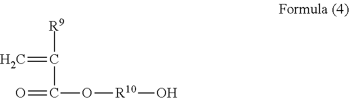

wherein R⁹ represents a hydrogen atom or a methyl group, and R¹⁰ represents an alkylene group having 2 or 3 carbon atoms.

By the copolymer according to the present invention, it is possible to provide a cell culture substrate (cell culture vessel) excellent in cellular adhesiveness regardless of a shape or design of a cell culture substrate (cell culture vessel).

In the present description, the alkoxyalkyl (meth)acrylate of the Formula (1) is simply referred to as “alkoxyalkyl (meth)acrylate”. Regarding the copolymer (a), the structural unit (a-1) derived from alkoxyalkyl (meth)acrylate of the Formula (1) is simply referred to as “structural unit (a-1)”, the trialkyl aminoalkyl (meth)acrylate of the Formula (2) is simply referred to as “trialkyl aminoalkyl (meth)acrylate”, the structural unit (a-2) derived from trialkyl aminoalkyl (meth)acrylate of the Formula (2) is simply referred to as “structural unit (a-2)”, and the copolymer (a) having the structural unit (a-1) and the structural unit (a-2) is also simply referred to as “copolymer (a)”. Similarly, regarding the copolymer (b), the structural unit (b-1) derived from alkoxyalkyl (meth)acrylate of the Formula (1) is simply referred to as “structural unit (b-1)”, the carboxyalkyl (meth)acrylate of the Formula (3) is simply referred to as “carboxyalkyl (meth)acrylate”, the structural unit (b-2) derived from carboxyalkyl (meth)acrylate of the Formula (3) is simply referred to as “structural unit (b-2)”, and the copolymer (b) having the structural unit (b-1) and the structural unit (b-2) is also simply referred to as “copolymer (b)”. Similarly, regarding the copolymer (c), the structural unit (c-1) derived from alkoxyalkyl (meth)acrylate of the Formula (1) is simply referred to as “structural unit (c-1),” the hydroxyalkyl (meth)acrylate of the Formula (4) is simply referred to as “hydroxyalkyl (meth)acrylate”, the structural unit (c-2) derived from hydroxyalkyl (meth)acrylate of the Formula (4) is simply referred to as “structural unit (c-2)”, and the copolymer (c) having the structural unit (c-1) and the structural unit (c-2) is also simply referred to as “copolymer (c)”.

In the present description, the copolymer (a), the copolymer (b), and the copolymer (c) may be collectively referred to as the “copolymer” or the “copolymer according to the present invention”.

Further, in the present description, the term “(meth)acrylate” includes both acrylate and methacrylate”. Similarly, the term “(meth)acrylic acid” includes both acrylic acid and methacrylic acid, and “(meth)acryloyl” includes both acryloyl and methacryloyl.

The cell culture substrate of the present invention has a feature in that a coating layer containing at least one of the copolymers (a) to (c) is formed on at least one surface of a polymer substrate. The coating layer (coating film, coating) formed by using the copolymers (a) to (c) has cellular adhesiveness equal to or more than that of the plasma treated substrate. Further, the coating film (coating layer) formed by using the copolymers (a) to (c) also has excellent cell proliferation ability (cell expanding ability). Here, the mechanism for exhibiting the effects by the present invention is presumed to be as follows. Incidentally, the present invention is not limited to the following presumption.

Conventionally, as a means for imparting cell adhesion, there has been a method of applying a cell adhesion factor such as fibronectin, laminin, or collagen to a substrate, a method of subjecting a substrate to treatment with plasma, gamma rays, or electrons, and the like. Of them, the former method has problems in that a cell adhesion factor is expensive and cannot be typically reused since the cell adhesion factor is a natural material, and the like. Further, in the latter method, the plasma treatment can impart particularly excellent cell adhesion to a substrate. Meanwhile, in recent years, a structure in which a porous body is inserted as a culture scaffold in a bag, a hollow fiber structure, a sponge structure, a flocculent (glass wool) structure, and a structure in which a plurality of dishes are laminated are used as a suitable culture scaffold. However, the latter method has a problem in that it is difficult or impossible to apply the method to such diversified and complicated structure. Currently, from the viewpoint that the complicated structures are excellent as a culture scaffold, there is a need for a means to provide a culture scaffold with cell adhesion comparable to or greater than that by plasma treatment.

In view of the circumstances, the present inventors have focused on the fact that a polymer is excellent in coating operability and have conducted intensive studies on a polymer excellent in cellular adhesiveness. It has been conventionally known that polyalkoxyalkyl (meth)acrylate such as polymethoxyethyl acrylate has cell adhesion. However, a coating film of polyalkoxyalkyl (meth)acrylate is inferior to a plasma treated substrate in cell adhesion (comparison between Comparative Example 6 and Reference Example 1 as described later). Further, the coating film of polyalkoxyalkyl (meth)acrylate is inferior in ability to expand cells on a substrate (cell expansion ability, cell proliferation activity). Therefore, the present inventors have evaluated cell adhesion of various copolymers obtained by copolymerization of various monomers with alkoxyalkyl (meth)acrylate, to find that a copolymer of alkoxyalkyl (meth)acrylate with trialkyl aminoalkyl (meth)acrylate, carboxyalkyl (meth)acrylate, or hydroxyalkyl (meth)acrylate at a specific composition has cell adhesion comparable to or greater than that by plasma treatment. Further, the present inventors have also found that these copolymers are excellent in cell proliferation activity (cell expansion ability). The detailed mechanism thereof is not clear but is presumed as follows: a quaternary ammonium salt (—N⁺R₃.X⁻) of trialkyl aminoalkyl (meth)acrylate, a carboxyl group (—COOH) of carboxyalkyl (meth)acrylate, or a hydroxyl group (—OH) of hydroxyalkyl (meth)acrylate is presumed to promote adhesion or expansion (proliferation) of cells via activation or induction of signal of cell-adhesion or cell-expansion (proliferation). Incidentally, for example, a homopolymer of hydroxyalkyl (meth)acrylate lowers cell adhesion. From this point, the founding of the present inventors that a copolymer of alkoxyalkyl (meth)acrylate with trialkyl aminoalkyl (meth)acrylate, carboxyalkyl (meth)acrylate, or hydroxyalkyl (meth)acrylate can improve cell adhesion as well as cell proliferation activity (cell expansion ability) as compared to a homopolymer of each monomer is very surprising.

Furthermore, according to the present invention, the coating layer can be formed by applying a solution obtained by dissolving the copolymers (a) to (c) in a solvent to a surface of a polymer substrate. Therefore, the coating layer (cell adhesion layer) can be easily formed even with respect to a cell culture substrate (cell culture vessel) having various shapes or designs.

Hereinafter, a preferred embodiment of the present invention will be described. Incidentally, the present invention is not limited only to the following embodiment.

In the present description, the term “X to Y” which indicates a range means the term “X or more and Y or less” including X and Y. Further, unless otherwise specified, operations and measurements of physical properties and the like are conducted under conditions of room temperature (20 to 25° C.)/relative humidity of 40 to 50% RH.

### <Cell Culture Substrate>

The cell culture substrate of the present invention comprises a coating layer containing at least one of copolymers (a), (b), and (c) formed on at least one surface of a polymer substrate. The coating layer containing the copolymer (a), (b), or (c) has equal to or more than that of plasma treatment. Further, the coating layer containing the copolymers (a), (b), or (c) is also excellent in cell expansion activity (cell proliferation activity). In addition, the coating layer can be simply formed in such a manner that the copolymer is dissolved in a solvent and the resultant solution is applied to a surface of the polymer substrate. Therefore, by using the copolymer according to the present invention, a coating layer (cell adhesion layer) having cellular adhesiveness (and further cell proliferation activity) can be formed on a surface of cell culture substrate (cell culture vessel) regardless of it shape or design.

[Copolymer (a)]

The copolymer (a) has 40% by mole or more and 90% by mole or less of a structural unit (a-1) derived from alkoxyalkyl (meth)acrylate of Formula (1) and 10% by mole or more and 60% by mole or less of a structural unit (a-2) derived from trialkyl aminoalkyl (meth)acrylate of Formula (2). Here, the total of the structural unit (a-1) and the structural unit (a-2) is 100% by mole.

The copolymer (a) has the structural unit (a-1), the structural unit (a-2), and as necessary, a structural unit derived from another monomer which will be described later in detail. Here, the arrangement of each structural unit is not particularly limited, but may be in the form of block (block copolymer), random (random copolymer), or alternate (alternate copolymer).

The alkoxyalkyl (meth)acrylate (structural unit (a-1)) imparts cell adhesion to a substrate. The trialkyl aminoalkyl (meth)acrylate (structural unit (a-2)) is presumed to promote cell adhesion by a quaternary ammonium salt (—N⁺R₃.X⁻) thereof. In addition, the trialkyl aminoalkyl (meth)acrylate (structural unit (a-2)) is presumed to impart cell expansion ability (cell proliferation activity) to the substrate by a quaternary ammonium salt (—N⁺R₃.X⁻) thereof. In particular, by combining the alkoxyalkyl (meth)acrylate (structural unit (a-1)) and the trialkyl aminoalkyl (meth)acrylate (structural unit (a-2)) at a specific ratio, more excellent cell adhesion than that of a homopolymer of alkoxyalkyl (meth)acrylate can be provided to the substrate, and the cell adhesion is comparable to or greater than that of a plasma-treated substrate. In addition thereto, by applying a solution of the copolymer (a) to a surface of a polymer substrate, a coating layer can be simply formed on a substrate having various shapes. Therefore, by using the copolymer (a), a coating layer (cell adhesion layer) having excellent cell adhesion (and further cell proliferation activity) can be formed on a cell culture substrate (cell culture vessel) having various shapes or designs.

The structural unit (a-1) constituting the copolymer (a) is contained at a ratio of 40% by mole or more and 90% by mole or less with respect to the total (100% by mole) of the structural unit (a-1) and the structural unit (a-2), and the structural unit (a-2) is contained at a ratio of 10% by mole or more and 60% by mole or less with respect to the total (100% by mole) of the structural unit (a-1) and the structural unit (a-2). Here, when the composition of the structural unit (a-2) is less than 10% by mole, the effects of the trialkyl aminoalkyl (meth)acrylate (structural unit (a-2)) (cell adhesion-promoting effect, and further cell proliferation activity-imparting effect) cannot be exhibited, and only cell adhesion similar to that of the homopolymer of alkoxyalkyl (meth)acrylate can be exhibited. On the other hand, when the composition of the structural unit (a-2) is more than 60% by mole, the effects by the introduction of alkoxyalkyl (meth)acrylate cannot be exhibited, and cell adhesion decreases as compared to that of the homopolymer of alkoxyalkyl (meth)acrylate (see Comparative Example 9 described later). From the viewpoint of further improving cell adhesion (and further cell proliferation activity), or the like, it is preferable that the structural unit (a-1) is contained at a ratio of 50% by mole or more and 70% by mole or less with respect to the total of the structural unit (a-1) and the structural unit (a-2), and the structural unit (a-2) is contained at a ratio of 30% by mole or more and 50% by mole or less with respect to the total of the structural unit (a-1) and the structural unit (a-2). It is more preferable that the structural unit (a-1) is contained at a ratio of 55% by mole or more and 65% by mole or less with respect to the total of the structural unit (a-1) and the structural unit (a-2), and the structural unit (a-2) is contained at a ratio of 35% by mole or more and 45% by mole or less with respect to the total of the structural unit (a-1) and the structural unit (a-2). That is, according to the preferred embodiment of the present invention, the copolymer (a) is a copolymer having 50% by mole or more and 70% by mole or less of the structural unit (a-1) derived from alkoxyalkyl (meth)acrylate of the Formula (1) and 30% by mole or more and 50% by mole or less of the structural unit (a-2) derived from trialkyl aminoalkyl (meth)acrylate of the Formula (2) (the total of the structural unit (a-1) and the structural unit (a-2) is 100% by mole). According to the more preferred embodiment of the present invention, the copolymer (a) is a copolymer having 55% by mole or more and 65% by mole or less of the structural unit (a-1) derived from alkoxyalkyl (meth)acrylate of the Formula (1) and 35% by mole or more and 45% by mole or less of the structural unit (a-2) derived from trialkyl aminoalkyl (meth)acrylate of the Formula (2) (the total of the structural unit (a-1) and the structural unit (a-2) is 100% by mole).

The copolymer (a) according to the present invention essentially includes the structural unit (a-1) and the structural unit (a-2), but may further have a structural unit derived from another monomer in addition to the structural unit (a-1) and the structural unit (a-2). Here, the another monomer is not particularly limited as long as it does not inhibit desired characteristics (cell adhesion and/or cell proliferation activity). Specific examples of the another monomer include acrylamide, N,N-dimethylacrylamide, N,N-diethylacrylamide, methacrylamide, N,N-dimethylmethacrylamide, N,N-dimethylmethacrylamide, ethylene, propylene, N-vinylacetamide, N-isopropenyl acetamide, N-(meth)acryloyl morpholine, and the like. A composition of the structural unit derived from another monomer in the case where the copolymer (a) further has a structural unit derived from the another monomer is not particularly limited as long as it does not inhibit desired characteristics (cell adhesion and cell proliferation activity), but the composition of the structural unit derived from another monomer is preferably more than 0% by mole and less than 10% by mole and more preferably about 3 to 8% by mole, with respect to the total of the structural unit (a-1) and the structural unit (a-2).

For the purpose of improving cell adhesion (and further cell proliferation activity), it is preferable that the copolymer (a) includes no structural units derived from another monomer, that is, the copolymer (a) according to the present invention is formed only of the structural unit (a-1) and the structural unit (a-2). That is, according to the preferred embodiment of the present invention, the copolymer (a) is composed of the structural unit (a-1) and the structural unit (a-2).

The structural unit (a-1) is derived from alkoxyalkyl (meth)acrylate of the following Formula (1). The structural unit (a-1) constituting the copolymer (a) may be used singly or in combination of two or more kinds. That is, the structural unit (a-1) may be composed only of sole structural unit derived from alkoxyalkyl (meth)acrylate of the following Formula (1), or may be composed of two or more structural units derived from alkoxyalkyl (meth)acrylate of the following Formula (1). In the latter case, each structural unit may be present in the form of block or random. Further, when the structural unit (a-1) is composed of two or more structural units derived from alkoxyalkyl (meth)acrylate of the following Formula (1), a composition of the structural unit (a-1) is a total ratio (molar ratio (% by mole)) of the structural units derived from alkoxyalkyl (meth)acrylate with respect to the total of the structural unit (a-1) and the structural unit (a-2).

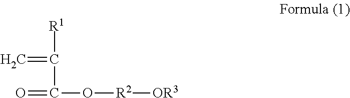

In the Formula (1), R¹ is a hydrogen atom or a methyl group. R² is an alkylene group having 2 or 3 carbon atoms. The alkylene group having 2 or 3 carbon atoms includes an ethylene group (—CH₂CH₂—), a trimethylene group (—CH₂CH₂CH₂—), and a propylene group (—CH(CH₃)CH₂— or —CH₂CH(CH₃)—). Among these, from the viewpoint of further improving cell adhesion (and further cell proliferation activity), or the like, R² preferably represents an ethylene group (—CH₂CH₂—) or a propylene group, and more preferably an ethylene group (—CH₂CH₂—). Further, R³ is an alkyl group having 1 to 3 carbon atoms. The alkyl group having 1 to 3 carbon atoms includes a methyl group, an ethyl group, a n-propyl group, and an isopropyl group. Among these, from the viewpoint of further improving cell adhesion (and further cell proliferation activity), or the like, R³ preferably represents a methyl group or an ethyl group, and more preferably a methyl group.

Specifically, examples of the alkoxyalkyl (meth)acrylate include methoxymethyl acrylate, methoxyethyl acrylate, methoxypropyl acrylate, ethoxymethyl acrylate, ethoxyethyl acrylate, ethoxypropyl acrylate, ethoxybutyl acrylate, propoxymethyl acrylate, butoxyethyl acrylate, methoxybutyl acrylate, methoxymethyl methacrylate, methoxyethyl methacrylate, ethoxymethyl methacrylate, ethoxyethyl methacrylate, propoxymethyl methacrylate, butoxyethyl methacrylate, and the like. Among these, from the viewpoint of further improving cell adhesion (and further cell proliferation activity), or the like, methoxyethyl (meth)acrylate, ethoxyethyl (meth)acrylate, or methoxybutyl (meth)acrylate is preferable, methoxyethyl (meth)acrylate is more preferable, and methoxyethyl acrylate (MEA) is particularly preferable.

The structural unit (a-2) is derived from trialkyl aminoalkyl (meth)acrylate of the following Formula (2). The structural unit (a-2) constituting the copolymer (a) may be used singly or in combination of two or more kinds. That is, the structural unit (a-2) may be composed only of sole structural unit derived from trialkyl aminoalkyl (meth)acrylate of the following Formula (2), or may be composed of two or more structural units derived from trialkyl aminoalkyl (meth)acrylate of the following Formula (2). In the latter case, each structural unit may be present in the form of block or random. Further, when the structural unit (a-2) is composed of two or more structural units derived from trialkyl aminoalkyl (meth)acrylate of the following Formula (2), a composition of the structural unit (a-2) is a total ratio (molar ratio (% by mole)) of the structural units derived from trialkyl aminoalkyl (meth)acrylate with respect to the total of the structural unit (a-1) and the structural unit (a-2).

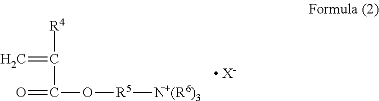

In the above Formula (2), R⁴ is a hydrogen atom or a methyl group. R⁵ is an alkylene group having 2 or 3 carbon atoms. The alkylene group having 2 or 3 carbon atoms includes an ethylene group (—CH₂CH₂—), a trimethylene group (—CH₂CH₂CH₂—), and a propylene group (—CH(CH₃)CH₂— or —CH₂CH(CH₃)—). Among these, from the viewpoint of further improving cell adhesion (and further cell proliferation activity), or the like, R⁵ preferably represents an ethylene group (—CH₂CH₂—) or a trimethylene group (—CH₂CH₂CH₂—), and more preferably an ethylene group (—CH₂CH₂—). Further, R⁶ each independently is an alkyl group having 1 to 3 carbon atoms. Here, a plurality of R⁶'s may be the same as or different from one another. The alkyl group having 1 to 3 carbon atoms includes a methyl group, an ethyl group, a n-propyl group, and an isopropyl group. Among these, from the viewpoint of further improving cell adhesion (and further cell proliferation activity), or the like, R⁶ preferably is a methyl group or an ethyl group, and more preferably a methyl group. Further, X⁻ represents an anion moiety. The anion moiety (counter anion) is not particularly limited as long as it can form a salt with the alkylammonium cation (—N⁺(R⁶)₃). Examples of such an anion include halide ions such as a fluoride ion, a chloride ion, a bromide ion, and an iodide ion; a hydrogen sulfate ion (HSO₄⁻); a sulfate ion (SO₄²⁻); a nitrate ion (NO₃⁻); a dihydrogenphosphate ion (H₂PO₄⁻), a hydrogen phosphate ion (HPO₄²⁻), a phosphate ion (PO₄³⁻); a perchlorate ion (ClO₄⁻); a hydroxide ion (OH⁻); carboxylate anions such as a citrate ion, an acetate ion, a malate ion, a fumarate ion, a lactate ion, a glutarate ion, and a maleate ion; and the like. From the viewpoint of solubility in a solvent of the copolymer, and the like, X⁻ (anion moiety, counter anion) is preferably selected from the group consisting of a halide ion, a hydrogen sulfate ion, a perchlorate ion, and a hydroxide ion, and is more preferably a halide ion.

Specifically, examples of the trialkyl aminoalkyl (meth)acrylate (excluding the anion moiety) include trimethylammonium ethyl acrylate, triethylammonium ethyl acrylate, tri-n-propylammonium ethyl acrylate, triisopropylammonium ethyl acrylate, trimethylammonium propyl acrylate, triethylammonium propyl acrylate, tri-n-propylammonium propyl acrylate, triisopropylammonium propyl acrylate, trimethylammonium isopropyl acrylate, triethylammonium isopropyl acrylate, tri-n-propylammonium isopropyl acrylate, and triisopropylammonium isopropyl acrylate, trimethylammonium ethyl methacrylate, triethylammonium ethyl methacrylate, tri-n-propylammonium ethyl methacrylate, triisopropylammonium ethyl methacrylate, trimethylammonium propyl methacrylate, triethylammonium propyl methacrylate, tri-n-propylammonium propyl methacrylate, triisopropylammonium propyl methacrylate, trimethylammonium isopropyl methacrylate, triethylammonium isopropyl methacrylate, tri-n-propylammonium isopropyl methacrylate, triisopropylammonium isopropyl methacrylate, and the like. Among these, from the viewpoint of further improving cell adhesion (and further cell proliferation activity), or the like, trimethylammonium ethyl (meth)acrylate (2-((meth)acryloyloxy)trimethylammonium) or triethylammonium ethyl (meth)acrylate is preferable and trimethylammonium ethyl (meth)acrylate (2-((meth)acryloyloxy)trimethylammonium) is more preferable.

A weight average molecular weight (Mw) of the copolymer (a) is not particularly limited, and is preferably 50,000 to 800,000. Within the above range, the solubility of the copolymer (a) in a solvent can be improved and application to a substrate can be uniformly conducted with ease. From the viewpoint of improving coating film formability, the weight average molecular weight of the copolymer (a) is more preferably 100,000 to 500,000 and particularly preferably 250,000 to 350,000. In the present description, as the “weight average molecular weight (Mw),” a value measured by gel permeation chromatography (GPC) using polystyrene as a standard and tetrahydrofuran (THF) as a mobile phase respectively is adopted. Specifically, the copolymer is dissolved in tetrahydrofuran (THF) so as to have a concentration of 10 mg/ml, thereby preparing a sample. Regarding the sample prepared as above, GPC column LF-804 (manufactured by Showa Denko K.K.) is attached to a GPC system LC-20 (manufactured by SHIMADZU CORPORATION), THF is supplied as a mobile phase, and polystyrene is used as a standard, to measure GPC of the copolymer. After preparing a calibration curve with polystyrene as standards, the weight average molecular weight (Mw) of the copolymer is calculated on the basis of the curve.

[Copolymer (b)]

The copolymer (b) has 85% by mole or more and 95% by mole or less of a structural unit (b-1) derived from alkoxyalkyl (meth)acrylate of Formula (1) and 5% by mole or more and 15% by mole or less of a structural unit (b-2) derived from carboxyalkyl (meth)acrylate of Formula (3). Here, the total of the structural unit (b-1) and the structural unit (b-2) is 100% by mole.

The copolymer (b) has the structural unit (b-1), the structural unit (b-2), and as necessary, a structural unit derived from another monomer which will be described later in detail. Here, the arrangement of each structural unit is not particularly limited, but may be in the form of block (block copolymer), random (random copolymer), or alternate (alternate copolymer).

The alkoxyalkyl (meth)acrylate (structural unit (b-1)) imparts cell adhesion to a substrate. The carboxyalkyl (meth)acrylate (structural unit (b-2)) is presumed to promote cell adhesion by a carboxyl group thereof. In addition, the carboxyalkyl (meth)acrylate (structural unit (b-2)) is presumed to impart cell expansion ability (cell proliferation activity) to the substrate by a carboxyl group thereof. In particular, by combining the alkoxyalkyl (meth)acrylate (structural unit (b-1)) and the carboxyalkyl (meth)acrylate (structural unit (b-2)) at a specific ratio, more excellent cell adhesion than that of a homopolymer of alkoxyalkyl (meth)acrylate can be provided to the substrate, and the cell adhesion is comparable to or greater than that of a plasma-treated substrate. In addition thereto, by applying a solution of the copolymer (b) to a surface of a polymer substrate, a coating layer can be simply formed on a substrate having various shapes. Therefore, by using the copolymer (b), a coating layer (cell adhesion layer) having excellent cell adhesion (and further cell proliferation activity) can be formed on a cell culture substrate (cell culture vessel) having various shapes or designs.

The structural unit (b-1) constituting the copolymer (b) contained at a ratio of 85% by mole or more and 95% by mole or less with respect to the total (100% by mole) of the structural unit (b-1) and the structural unit (b-2), and the structural unit (b-2) is contained at a ratio of 5% by mole or more and 15% by mole or less with respect to the total (100% by mole) of the structural unit (b-1) and the structural unit (b-2). Here, when the composition of the structural unit (b-2) is less than 5% by mole, the effects of the carboxyalkyl (meth)acrylate (structural unit (b-2)) (cell adhesion-promoting effect, and further cell proliferation activity-imparting effect) cannot be exhibited, and only cell adhesion similar to that of the homopolymer of alkoxyalkyl (meth)acrylate can be exhibited. On the other hand, when the composition of the structural unit (b-2) is more than 15% by mole, the effects by the introduction of alkoxyalkyl (meth)acrylate cannot be exhibited, and cell adhesion similar to that of the homopolymer of alkoxyalkyl (meth)acrylate is only exhibited (see Comparative Example 8 described later). From the viewpoint of further improving cell adhesion (and further cell proliferation activity), or the like, it is preferable that the structural unit (b-1) is contained at a ratio of 90% by mole or more and 95% by mole or less with respect to the total of the structural unit (b-1) and the structural unit (b-2), and the structural unit (b-2) is contained at a ratio of 5% by mole or more and 10% by mole or less with respect to the total of the structural unit (b-1) and the structural unit (b-2). It is more preferable that the structural unit (b-1) is contained at a ratio of more than 90% by mole and 95% by mole or less with respect to the total of the structural unit (b-1) and the structural unit (b-2), and the structural unit (b-2) is contained at a ratio of 5% by mole or more and less than 10% by mole with respect to the total of the structural unit (b-1) and the structural unit (b-2). That is, according to the preferred embodiment of the present invention, the copolymer (b) is a copolymer having 90% by mole or more and 95% by mole or less of the structural unit (b-1) derived from alkoxyalkyl (meth)acrylate of the Formula (1) and 5% by mole or more and 10% by mole or less of the structural unit (b-2) derived from carboxyalkyl (meth)acrylate of the Formula (3) (the total of the structural unit (b-1) and the structural unit (b-2) is 100% by mole). According to the more preferred embodiment of the present invention, the copolymer (b) is a copolymer having more than 90% by mole and 95% by mole or less of the structural unit (b-1) derived from alkoxyalkyl (meth)acrylate of the Formula (1) and 5% by mole or more and less than 10% by mole of the structural unit (b-2) derived from carboxyalkyl (meth)acrylate of the Formula (3) (the total of the structural unit (b-1) and the structural unit (b-2) is 100% by mole).

The copolymer (b) according to the present invention essentially includes the structural unit (b-1) and the structural unit (b-2), but may further have a structural unit derived from another monomer in addition to the structural unit (b-1) and the structural unit (b-2). Here, the another monomer is not particularly limited as long as it does not inhibit desired characteristics (cell adhesion and/or cell proliferation activity). Specifically, examples of the another monomer may be the same as those in the copolymer (a) described above. A composition of the structural unit derived from another monomer in the case where the copolymer (b) further has a structural unit derived from the another monomer is not particularly limited as long as it does not inhibit desired characteristics (cell adhesion and cell proliferation activity), but the composition of the structural unit derived from another monomer is preferably more than 0% by mole and less than 10% by mole and more preferably about 3 to 8% by mole, with respect to the total of the structural unit (b-1) and the structural unit (b-2).

For the purpose of improving cell adhesion (and further cell proliferation activity), it is preferable that the copolymer (b) includes no structural units derived from another monomer, that is, the copolymer (b) according to the present invention is formed only of the structural unit (b-1) and the structural unit (b-2). That is, according to the preferred embodiment of the present invention, the copolymer (b) is composed of the structural unit (b-1) and the structural unit (b-2).

The structural unit (b-1) is derived from alkoxyalkyl (meth)acrylate of the following Formula (1). Incidentally, the structural unit (b-1) constituting the copolymer (b) may be used singly or in combination of two or more kinds. That is, the structural unit (b-1) may be composed only of sole structural unit derived from alkoxyalkyl (meth)acrylate of the following Formula (1), or may be composed of two or more structural units derived from alkoxyalkyl (meth)acrylate of the following Formula (1). In the latter case, each structural unit may be present in the form of block or random. Further, when the structural unit (b-1) is composed of two or more structural units derived from alkoxyalkyl (meth)acrylate of the following Formula (1), a composition of the structural unit (b-1) is a total ratio (molar ratio (% by mole)) of the structural units derived from alkoxyalkyl (meth)acrylate with respect to the total of the structural unit (b-1) and the structural unit (b-2). Here, the detailed descriptions of Formula (1) and alkoxyalkyl (meth)acrylate are as defined in the copolymer (a), and thus the descriptions thereof will be omitted herein.

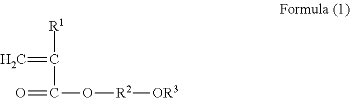

The structural unit (b-2) is derived from carboxyalkyl (meth)acrylate of the following Formula (3). Incidentally, the structural unit (b-2) constituting the copolymer (b) may be used singly or in combination of two or more kinds. That is, the structural unit (b-2) may be composed only of sole structural unit derived from carboxyalkyl (meth)acrylate of the following Formula (3), or may be composed of two or more structural units derived from carboxyalkyl (meth)acrylate of the following Formula (3). In the latter case, each structural unit may be present in the form of block or random. Further, when the structural unit (b-2) is composed of two or more structural units derived from carboxyalkyl (meth)acrylate of the following Formula (3), a composition of the structural unit (b-2) is a total ratio (molar ratio (% by mole)) of the structural units derived from carboxyalkyl (meth)acrylate with respect to the total of the structural unit (b-1) and the structural unit (b-2).

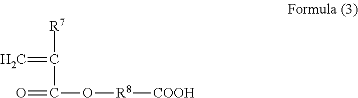

In the above Formula (3), R⁷ is a hydrogen atom or a methyl group. R⁸ is an alkylene group having 2 or 3 carbon atoms. The alkylene group having 2 or 3 carbon atoms includes an ethylene group (—CH₂CH₂—), a trimethylene group (—CH₂CH₂CH₂—), and a propylene group (—CH(CH₃)CH₂— or —CH₂CH(CH₃)—). Among these, from the viewpoint of further improving cell adhesion (and further cell proliferation activity), or the like, R⁵ preferably represents an ethylene group (—CH₂CH₂—) or a trimethylene group (—CH₂CH₂CH₂—), and more preferably an ethylene group (—CH₂CH₂—).

Specifically, examples of the carboxyalkyl (meth)acrylate include carboxyethyl acrylate, carboxypropyl acrylate, carboxyisopropyl acrylate, carboxyethyl methacrylate, carboxy propyl methacrylate, carboxyisopropyl methacrylate, and the like. Among these, from the viewpoint of further improving cell adhesion (and further cell proliferation activity), or the like, carboxyethyl (meth)acrylate is preferable, and carboxyethyl acrylate (CEA) is more preferable.

A weight average molecular weight (Mw) of the copolymer (b) is not particularly limited, and is preferably 50,000 to 800,000. Within the above range, the solubility of the copolymer (b) in a solvent can be improved and application to a substrate can be uniformly conducted with ease. From the viewpoint of improving coating film formability, the weight average molecular weight of the copolymer (b) is more preferably 100,000 to 500,000 and particularly preferably 150,000 to 250,000.

[Copolymer (c)]

The copolymer (c) has 70% by mole or more and 90% by mole or less of a structural unit (c-1) derived from alkoxyalkyl (meth)acrylate of Formula (1) and 10% by mole or more and 30% by mole or less of a structural unit (c-2) derived from hydroxyalkyl (meth)acrylate of Formula (4). Here, the total of the structural unit (c-1) and the structural unit (c-2) is 100% by mole.

The copolymer (c) has the structural unit (c-1) and the structural unit (c-2), and as necessary, a structural unit derived from another monomer which will be described later in detail. Here, the arrangement of each structural unit is not particularly limited, but may be in the form of block (block copolymer), random (random copolymer), or alternate (alternate copolymer).

The alkoxyalkyl (meth)acrylate (structural unit (c-1)) imparts cell adhesion to a substrate. The hydroxyalkyl (meth)acrylate (structural unit (c-2)) is presumed to promote cell adhesion by a hydroxyl group thereof. In addition, the hydroxyalkyl (meth)acrylate (structural unit (c-2)) is presumed to impart cell expansion ability (cell proliferation activity) to the substrate by a hydroxyl group thereof. In particular, by combining the alkoxyalkyl (meth)acrylate (structural unit (c-1)) and the hydroxyalkyl (meth)acrylate (structural unit (c-2)) at a specific ratio, more excellent cell adhesion than that of a homopolymer of alkoxyalkyl (meth)acrylate can be provided to the substrate, and the cell adhesion is comparable to or greater than that of a plasma-treated substrate. In addition thereto, by applying a solution of the copolymer (c) to a surface of a polymer substrate, a coating layer can be simply formed on a substrate having various shapes. Therefore, by using the copolymer (c), a coating layer (cell adhesion layer) having excellent cell adhesion (and further cell proliferation activity) can be formed on a cell culture substrate (cell culture vessel) having various shapes or designs.

The structural unit (c-1) constituting the copolymer (c) is 70% by mole or more and 90% by mole or less with respect to the total (100% by mole) of the structural unit (c-1) and the structural unit (c-2), and the structural unit (c-2) is 10% by mole or more and 30% by mole or less with respect to the total (100% by mole) of the structural unit (c-1) and the structural unit (c-2). Here, when the composition of the structural unit (c-2) is less than 10% by mole, the effects of the hydroxyalkyl (meth)acrylate (structural unit (c-2)) (cell adhesion-promoting effect, and further cell proliferation activity-imparting effect) cannot be exhibited, and only cell adhesion similar to that of the homopolymer of alkoxyalkyl (meth)acrylate can be exhibited. On the other hand, when the composition of the structural unit (c-2) is more than 30% by mole, the effects by the introduction of alkoxyalkyl (meth)acrylate cannot be exhibited, and cell adhesion decreases as compared to that of the homopolymer of alkoxyalkyl (meth)acrylate (see Comparative Example 7 described later). From the viewpoint of further improving cell adhesion (and further cell proliferation activity), or the like, it is preferable that the structural unit (c-1) is contained at a ratio of 80% by mole or more and 90% by mole or less with respect to the total of the structural unit (c-1) and the structural unit (c-2), and the structural unit (c-2) is contained at a ratio of 10% by mole or more and 20% by mole or less with respect to the total of the structural unit (c-1) and the structural unit (c-2). It is more preferable that the structural unit (c-1) is contained at a ratio of more than 80% by mole and 90% by mole or less with respect to the total of the structural unit (c-1) and the structural unit (c-2), and the structural unit (c-2) is contained at a ratio of 10% by mole or more and less than 20% by mole with respect to the total of the structural unit (c-1) and the structural unit (c-2). That is, according to the preferred embodiment of the present invention, the copolymer (c) is a copolymer having 80% by mole or more and 90% by mole or less of the structural unit (c-1) derived from alkoxyalkyl (meth)acrylate of the Formula (1) and 10% by mole or more and 20% by mole or less of the structural unit (c-2) derived from hydroxyalkyl (meth)acrylate of the Formula (4) (the total of the structural unit (c-1) and the structural unit (c-2) is 100% by mole). According to the more preferred embodiment of the present invention, the copolymer (c) is a copolymer having more than 80% by mole and 90% by mole or less of the structural unit (c-1) derived from alkoxyalkyl (meth)acrylate of the Formula (1) and 10% by mole or more and less than 20% by mole of the structural unit (c-2) derived from hydroxyalkyl (meth)acrylate of the Formula (4) (the total of the structural unit (c-1) and the structural unit (c-2) is 100% by mole).

The copolymer (c) according to the present invention essentially includes the structural unit (c-1) and the structural unit (c-2), but may further have a structural unit derived from another monomer in addition to the structural unit (c-1) and the structural unit (c-2). Here, the another monomer is not particularly limited as long as it does not inhibit desired characteristics (cell adhesion and/or cell proliferation activity). Specifically, examples of the another monomer may be the same as those in the copolymer (a) described above. A composition of the structural unit derived from another monomer in the case where the copolymer (c) further has a structural unit derived from the another monomer is not particularly limited as long as it does not inhibit desired characteristics (cell adhesion and cell proliferation activity), but the composition of the structural unit derived from another monomer is preferably more than 0% by mole and less than 10% by mole and more preferably about 3 to 8% by mole, with respect to the total of the structural unit (c-1) and the structural unit (c-2).

For the purpose of improving cell adhesion (and further cell proliferation activity), it is preferable that the copolymer (c) includes no structural units derived from another monomer, that is, the copolymer (c) according to the present invention is formed only of the structural unit (c-1) and the structural unit (c-2). That is, according to the preferred embodiment of the present invention, the copolymer (c) is composed of the structural unit (c-1) and the structural unit (c-2).

The structural unit (c-1) is derived from alkoxyalkyl (meth)acrylate of the following Formula (1). Incidentally, the structural unit (c-1) constituting the copolymer (c) may be used singly or in combination of two or more kinds. That is, the structural unit (c-1) may be composed only of sole structural unit derived from alkoxyalkyl (meth)acrylate of the following Formula (1), or may be composed of two or more structural units derived from alkoxyalkyl (meth)acrylate of the following Formula (1). In the latter case, each structural unit may be present in the form of block or random. Further, when the structural unit (c-1) is composed of two or more structural units derived from alkoxyalkyl (meth)acrylate of the following Formula (1), a composition of the structural unit (c-1) is a total ratio (molar ratio (% by mole)) of the structural units derived from alkoxyalkyl (meth)acrylate with respect to the total of the structural unit (c-1) and the structural unit (c-2). Here, the detailed descriptions of Formula (1) and alkoxyalkyl (meth)acrylate are as defined in the copolymer (a), and thus the descriptions thereof will be omitted herein.

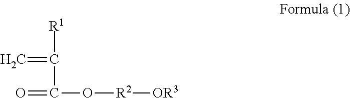

The structural unit (c-2) is derived from hydroxyalkyl (meth)acrylate of the following Formula (4). Incidentally, the structural unit (c-2) constituting the copolymer (c) may be used singly or in combination of two or more kinds. That is, the structural unit (c-2) may be composed only of sole structural unit derived from hydroxyalkyl (meth)acrylate of the following Formula (4), or may be composed of two or more structural units derived from hydroxyalkyl (meth)acrylate of the following Formula (4). In the latter case, each structural unit may be present in the form of block or random. Further, when the structural unit (c-2) is composed of two or more structural units derived from hydroxyalkyl (meth)acrylate of the following Formula (4), a composition of the structural unit (c-2) is a total ratio (molar ratio (% by mole)) of the structural units derived from hydroxyalkyl (meth)acrylate with respect to the total of the structural unit (c-1) and the structural unit (c-2).

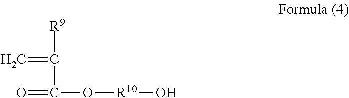

In the above Formula (4), R⁹ is a hydrogen atom or a methyl group. R¹⁰ is an alkylene group having 2 or 3 carbon atoms. The alkylene group having 2 or 3 carbon atoms includes an ethylene group (—CH₂CH₂—), a trimethylene group (—CH₂CH₂CH₂—), and a propylene group (—CH(CH₃)CH₂— or —CH₂CH(CH₃)—). Among these, from the viewpoint of further improving cell adhesion (and further cell proliferation activity), or the like, R⁵ preferably represents an ethylene group (—CH₂CH₂—) or a trimethylene group (—CH₂CH₂CH₂—), and more preferably an ethylene group (—CH₂CH₂—).

Specifically, examples of hydroxyalkyl (meth)acrylate include hydroxyethyl acrylate, hydroxypropyl acrylate, hydroxyisopropyl acrylate, hydroxyethyl methacrylate, hydroxypropyl methacrylate, hydroxyisopropyl methacrylate, and the like. Among these, from the viewpoint of further improving cell adhesion (and further cell proliferation activity), or the like, hydroxyethyl (meth)acrylate is preferable, and hydroxyethyl methacrylate (HEMA) is more preferable.

A weight average molecular weight (Mw) of the copolymer (c) is not particularly limited, and is preferably 50,000 to 800,000. Within the above range, the solubility of the copolymer (c) in a solvent can be improved and application to a substrate can be uniformly conducted with ease. From the viewpoint of improving coating film formability, the weight average molecular weight of the copolymer (c) is more preferably 100,000 to 500,000 and particularly preferably 150,000 to 300,000.

As described above, the coating layer according to the present invention contains at least one of the copolymers (a) to (c), but from the viewpoint of further improvement in cell adhesion (and further cell proliferation activity), and the like, the coating layer preferably contains at least one of the copolymer (c) and the copolymer (b). Further, from the viewpoint of subculture (particularly, cell adhesion of cells at the time of subculture after removing cultured cells from a substrate), the coating layer preferably contains at least one of the copolymer (a) and the copolymer (b).

The copolymer according to the present invention can be produced by employing a conventionally known polymerization method such as bulk polymerization, suspension polymerization, emulsion polymerization, solution polymerization, living radical polymerization method, polymerization method using a macroinitiator, polycondensation method, or the like, for example, although not particularly limited thereto. Specifically, in a case where the copolymer according to the present invention is a block copolymer, for example, a living radical polymerization method or a polymerization method using a macroinitiator is preferably used. As the living radical polymerization method, although not particularly limited thereto, a method described in JP H11-263819 A, JP 2002-145971 A, JP 2006-316169 A, or the like, an atom transfer radical polymerization (ATRP) method, and the like can be applied similarly or appropriately modified, for example.

Alternatively, for example, in a case where the copolymer according to the present invention is a random copolymer, it is preferable to use a method of stirring the alkoxyalkyl (meth)acrylate of the Formula (1), a desired monomer (at least one of the trialkyl aminoalkyl (meth)acrylate of the Formula (2), the carboxyalkyl (meth)acrylate of the Formula (3), and the hydroxyalkyl (meth)acrylate of the Formula (4)), and as necessary, one or two or more kinds of monomer which is copolymerizable with those components (another monomer, copolymerizable monomer), in a polymerization solvent, with a polymerization initiator to prepare a monomer solution, and heating the monomer solution to perform copolymerization. In the method, a polymerization solvent which can be used in the preparation of the monomer solution is not particularly limited as long as it can dissolve the monomer used above. Examples thereof include aqueous solvents such as water, alcohols such as methanol, ethanol, propanol, and isopropanol, and polyethylene glycols; aromatic solvents such as toluene, xylene, and tetralin; halogen-based solvents such as chloroform, dichloroethane, chlorobenzene, dichlorobenzene, and trichlorobenzene; and the like. Among these, taking in consideration of easy dissolution of the monomer, or the like, methanol is preferable. Further, a concentration of the monomer in the monomer solution is not particularly limited, but the concentration of the monomer in the monomer solution is typically 15 to 60% by weight, more preferably 20 to 50% by weight, and particularly preferably 25 to 45% by weight. Incidentally, the concentration of the monomer means a total concentration of the alkoxyalkyl (meth)acrylate of the Formula (1) and a desired monomer (at least one of the trialkyl aminoalkyl (meth)acrylate of the Formula (2), the carboxyalkyl (meth)acrylate of the Formula (3), and the hydroxyalkyl (meth)acrylate of the Formula (4)), and if being used, a monomer which is copolymerizable with those components (another monomer, copolymerizable monomer).

The polymerization initiator is not particularly limited, and a known polymerization initiator may be used. From the viewpoint of high polymerization stability, the polymerization initiator is preferably a radical polymerization initiator. Specific examples thereof include persulfates such as potassium persulfate (KPS), sodium persulfate, and ammonium persulfate; peroxides such as hydrogen peroxide, t-butyl peroxide, and methyl ethyl ketone peroxide; and azo compounds such as azobisisobutyronitrile (AIBN), 2,2′-azobis(4-methoxy-2,4-dimethylvaleronitrile), 2,2′-azobis(2,4-dimethylvaleronitrile), 2,2′-azobis[2-(2-imidazoline-2-yl)propane]dihydrochloride, 2,2′-azobis[2-(2-imidazoline-2-yl)propane]disulfate dihydrate, 2,2′-azobis(2-methylpropionamidine)dihydrochloride, 2,2′-azobis[N-(2-carboxyethyl)-2-methylpropionamidine)]hydrate, 3-hydroxy-1,1-dimethylbutyl peroxyneodecanoate, α-cumylperoxy neodecanoate, 1,1,3,3-tetrabutyl peroxyneodecanoate, t-butyl peroxyneodecanoate, t-butyl peroxyneoheptanoate, t-butyl peroxypivalate, t-amyl peroxyneodecanoate, t-amyl peroxypivalate, di(2-ethylhexyl)peroxydicarbonate, di(secondary butyl)peroxydicarbonate, and azobiscyanovaleric acid. Further, for example, a reducing agent such as sodium sulfite, sodium hydrogen sulfite, or ascorbic acid may be used in combination with the radical polymerization initiator as a redox type initiator. A blending amount of the polymerization initiator is preferably 0.0005 to 0.005 mol with respect to 1 mol of a total amount of the monomers. With such a blending amount of the polymerization initiator, copolymerization of the respective monomers can efficiently proceed.

The polymerization initiator as it is may be mixed with the alkoxyalkyl (meth)acrylate of the Formula (1), a desired monomer (at least one of the trialkyl aminoalkyl (meth)acrylate of the Formula (2), the carboxyalkyl (meth)acrylate of the Formula (3), and the hydroxyalkyl (meth)acrylate of the Formula (4)), and if being used, a monomer which is copolymerizable with those components (another monomer, copolymerizable monomer; the same applies hereinafter), and a polymerization solvent, or alternatively a solution of the polymerization initiator obtained by being dissolved in another solvent in advance may be mixed with the monomers and the polymerization solvent. In the latter case, another solvent used to dissolve the polymerization initiator is not particularly limited as long as it can dissolve the polymerization initiator, but the same solvent as the polymerization solvent described above can be exemplified. Further, another solvent may be the same as or different from the polymerization solvent, but in consideration of easy control of polymerization, and the like, the same solvent as the polymerization solvent is preferably used. Further, in this case, a concentration of the polymerization initiator in another solvent is not particularly limited, but in consideration of easy mixing, and the like, the addition amount of the polymerization initiator is preferably 0.1 to 10 parts by weight and more preferably 0.5 to 5 parts by weight, with respect to 100 parts by weight of another solvent.

Further, in the case of using the polymerization initiator in the solution state, deaeration treatment may be performed in advance before adding a solution in which the monomers (alkoxyalkyl (meth)acrylate, a desired monomer, and a copolymerizable monomer which is used as necessary) are dissolved in the polymerization solvent, to the polymerization initiator solution. For the deaeration treatment, for example, the solution may be bubbled with an inert gas such as nitrogen gas or argon gas for about 0.5 to 5 hours. In the deaeration treatment, the solution may be adjusted to about 30° C. to 80° C., preferably to a polymerization temperature in a polymerization step as described below.

Next, the monomer solution is heated to copolymerize the respective monomers. Here, as the copolymerization method, for example, a known polymerization method such as radical polymerization, anionic polymerization, or cationic polymerization can be adopted, and radical polymerization which facilitates production is preferably used.

The polymerization conditions are not particularly limited as long as the alkoxyalkyl (meth)acrylate of the Formula (1) and a desired monomer (at least one of the trialkyl aminoalkyl (meth)acrylate of the Formula (2), the carboxyalkyl (meth)acrylate of the Formula (3), and the hydroxyalkyl (meth)acrylate of the Formula (4)), and if being used, a monomer which is copolymerizable with those components (another monomer, copolymerizable monomer) can be copolymerized. Specifically, the copolymerization temperature is preferably 30 to 80° C. and more preferably 40° C. to 55° C. Further, the copolymerization time is preferably is 1 to 24 hours and more preferably 5 to 12 hours. Under such conditions, copolymerization of the respective monomers can efficiently proceed. Further, it is possible to effectively suppress or prevent gelation in the polymerization step and to achieve high production efficiency.

As necessary, a chain transfer agent, a polymerization rate-adjusting agent, a surfactant, and other additives may be appropriately used during the polymerization.

An atmosphere under which the polymerization reaction is carried out is not particularly limited, and the reaction can be carried out under an air atmosphere, an inert gas atmosphere such as nitrogen gas or argon gas, and the like. Further, during the polymerization reaction, the reaction solution may be stirred.

The polymer after polymerization can be purified by a general purification method such as a reprecipitation method (precipitation method), a dialysis method, an ultrafiltration method, or an extraction method.

The purified polymer can be dried by an arbitrary method such as freeze drying, vacuum drying, spray drying, or heat drying, but freeze drying or vacuum drying is preferred from the viewpoint that the physical properties of the polymer are less affected.

### [Polymer Substrate]

In the present invention, a coating layer containing at least one of the copolymers (a) to (c) according to the present invention is formed on at least one surface of the polymer substrate. Herein, the coating layer is formed on at least a surface of the polymer substrate with which cells contact (for example, on which a liquid containing cells flows or cells are cultured). Further, it is not necessary to form the coating layer on an entire surface of the polymer substrate. The coating layer may be formed on a portion (a part) of the surface of the polymer substrate with which cells contact (for example, on which a liquid containing cells flows or cells are cultured). From the viewpoint of further improving cell adhesion (and further cell proliferation activity), the coating layer is preferably formed on the entire surface of the polymer substrate at the side with which cells contact (for example, on which a liquid containing cells flows or cells are cultured).

Herein, a structure of the polymer substrate is not limited. In addition to the plane structure, the polymer substrate can be designed in various structures (forms) such as a structure in which a porous body is inserted, a hollow fiber structure, a porous membrane structure, a sponge structure, a flocculent (glass wool) structure. As described later, the cell culture substrate of the present invention can be suitably used in a bioreactor, particularly, a hollow fiber type bioreactor. Therefore, the polymer substrate preferably has hollow fibers and is more preferably a porous membrane formed of a plurality of hollow fibers. That is, according to the preferred embodiment of the present invention, the polymer substrate is a porous membrane. In the case where the polymer substrate is a porous membrane, an inner diameter (diameter) of the hollow fiber constituting the porous membrane is not particularly limited, but is preferably 50 to 1,000 μm, more preferably 100 to 500 μm, and particularly preferably about 150 to 350 μm. An outer diameter (diameter) of the hollow fiber constituting the porous membrane is not particularly limited, but is preferably 100 to 1,200 μm, more preferably 150 to 700 μm, and particularly preferably about 200 to 500 μm. A length of the hollow fiber constituting the porous membrane when the polymer substrate is a porous membrane is not particularly limited, but is preferably 50 to 900 mm, more preferably 100 to 700 mm, and particularly preferably about 150 to 500 mm. The number of the hollow fibers constituting the porous membrane when the polymer substrate is a porous membrane is not particularly limited, but is, for example, about 1,000 to 100,000, more preferably 3,000 to 50,000, and particularly preferably about 5,000 to 25,000. In an embodiment, the polymer substrate is configured by about 9,000 hollow fibers having an average length of about 295 mm, an average inner diameter of 215 μm, and an average outer diameter of 315 μm. Herein, the coating layer may be formed on the inner side or the outer side of the hollow fiber membrane, but is preferably formed on the inner (lumen) surface.

A method for producing a hollow fiber and a porous membrane is not particularly limited, and a known production method can be applied similarly or appropriately modified. For example, it is preferable that micro fine holes are formed on a wall of hollow fiber by a stretching method or a solid-liquid phase separation method.

A material constituting the polymer substrate is also not particularly limited. Specific examples thereof include a hydrophobic polymer material such as polystyrene, polysulfone, polyacrylonitrile, polytetrafluoroethylene, or cellulose acetate, and the like. Further, the polymer substrate may be produced by a semi-permeable, biocompatible polymer material such as a blend of polyamide, polyarylethersulfone, and polyvinylpyrrolidone (PA/PAES/PVP). Such a semi-permeable membrane allows transfer of nutrient, waste, and dissolved gas through the membrane between the extracapillary (EC) space of the hollow fiber and the intracapillary (IC) space of the hollow fiber. The molecule transfer characteristics of the hollow fiber membrane may be selected such that a metabolic waste product can pass through the membrane to be dispersed into a hollow fiber lumen and then removed therefrom, and at the same time, loss of an expensive reagent (such as a growth factor or cytokine) necessary for cell growth from the hollow fiber can be minimized. In a case where the polymer substrate is hollow fibers formed of PA/PAES/PVP, an outer layer of the hollow fiber may have an open pore structure with a certain surface roughness. An opening (diameter) of the pore is not particularly limited, but is in the range of about 0.5 to about 3 μm, and the number of pores on the outer surface of the hollow fiber may be in the range of about 10,000 to about 150,000 per 1 square millimeter (1 mm²). A thickness of the outer layer of the hollow fiber is not particularly limited, and for example, is in the range of about 1 to about 10 μm. The hollow fiber may have an additional layer (second layer) on the outer side, and at this time, the additional layer (second layer) preferably has a sponge structure having a thickness of about 1 to about 15 μm. The second layer having such a structure can serve as a support for the outer layer. Further, in this embodiment, the hollow fiber may have a further additional layer (third layer) at the outer side of the second layer. In this embodiment, the further additional layer (third layer) preferably has a finger-like structure. With the third layer having such a structure, mechanical stability is obtainable. Further, a high void volume with low resistance to membrane transfer of molecules can be provided. In this embodiment, during use, the finger-like voids are filled with a fluid and the fluid lowers resistance for diffusion and convection as compared with a matrix with a sponge-filled structure having a lower void volume. This third layer has a thickness of, preferably, about 20 to about 60 μm.

Further, the polymer substrate may have about 65% by weight to about 95% by weight of at least a hydrophobic polymer and about 5% by weight to about 35% by weight of at least a hydrophilic polymer. At this time, a total amount of the hydrophobic polymer and the hydrophilic polymer is 100% by weight. Here, the hydrophobic polymer is not particularly limited, and examples thereof include polyamide (PA), polyaramide (PAA), polyarylethersulfone (PAES), polyethersulphone (PES), polysulfone (PSU), polyarylsulphone (PASU), polycarbonate (PC), polyether, polyurethane (PUR), polyetherimide, and polyethersulphone; a mixture of polyarylethersulfone and polyamide; and the like. These hydrophobic polymers may be used singly or as a mixture of two or more kinds thereof. Further, the hydrophilic polymer is not particularly limited, and examples thereof include polyvinylpyrrolidone (PVP), polyethylene glycol (PEG), polyglycolmonoester, water soluble cellulosic derivatives, polysorbate, polyethylene-polypropylene oxide copolymers, and the like. These hydrophilic polymers may be used singly or as a mixture of two or more kinds thereof.

A method of forming a coating layer containing the copolymer according to the present invention on a surface of the polymer substrate is not particularly limited. For example, in a case where the surface of the polymer substrate has a flat dish (plate) structure, a method of applying a copolymer-containing solution obtained by dissolving the copolymer according to the present invention to a predetermined surface (for example, by adding to a well) and then drying coating film can be used. Further, for example, in a case where the polymer substrate is a hollow fiber or a porous membrane, a method of bringing a copolymer-containing solution obtained by dissolving the copolymer according to the present invention into contact with a cell contact portion of the hollow fiber (for example, by flowing on an inner surface (lumen) or an outer surface of the hollow fiber) and then drying coating film can be used. Incidentally, in a case where the polymer substrate is a porous membrane formed by a plurality of hollow fibers, coating with a copolymer-containing solution may be performed with respect to one hollow fiber and then the hollow fibers may be bundled, or a plurality of hollow fibers are bundled to produce a porous membrane and then the coating may be performed.

Herein, a solvent for dissolving the copolymer according to the present invention is not particularly limited as long as it can dissolve the copolymer according to the present invention. From the viewpoint of solubility of the copolymer, and the like, for example, aqueous solvents such as water, alcohols such as methanol or propanol, and polyethylene glycols; ketone-based solvents such as acetone; furan-based solvents such as tetrahydrofuran; and the like are exemplified. The solvent may be used singly or in the form of a mixture of two or more kinds thereof. Among these, in consideration of further improvement in solubility of the copolymer according to the present invention, the solvent is preferably methanol. A concentration of the copolymer in the copolymer-containing solution is not particularly limited. In consideration of the easy application to the substrate, the effects of reducing coating unevenness, and the like, the concentration thereof is preferably 0.0001 to 5% by weight and more preferably 0.001 to 1% by weight.

Further, a method of coating the copolymer is not particularly limited, and a conventionally known method such as filling, dip coating (immersion method), spraying, spin coating, dropping, doctor blade, brush coating, roll coater, air knife coating, curtain coating, wire bar coating, gravure coating, or mixed solution-impregnated sponge coating can be applied.

Further, conditions for forming the coating film of the copolymer are not particularly limited. For example, a contact time of the copolymer-containing solution and the polymer substrate (for example, a time for circulating the copolymer-containing solution to a lumen or an outer surface of the hollow fiber) is preferably 1 to 5 minutes and more preferably 1 to 3 minutes, in consideration of the easy formation of the coating film (thus coating layer), the effect of reducing coating unevenness, and the like. Further, a contact temperature of the copolymer-containing solution and the polymer substrate (for example, a temperature at which the copolymer-containing solution is circulated to a lumen or an outer surface of hollow fiber) is preferably 5 to 40° C. and more preferably 15 to 30° C., in consideration of the easy formation of the coating film (thus coating layer), the effect of reducing coating unevenness, and the like.

An amount of the copolymer-containing solution applied to a surface of the polymer substrate is not particularly limited, but is preferably such an amount that a thickness of the coating layer after drying is about 5 nm to 20 μm. Incidentally, in a case where such a thickness cannot be obtainable by single contact (application), a contact (application) step (or the application step and a drying step described later) may be repeated until a desired thickness is obtainable.

Next, by drying the coating film after the contact of the polymer substrate and the copolymer-containing solution, the coating layer (coating film) by the copolymer according to the present invention is formed on the surface of the polymer substrate. Herein, drying conditions are not particularly limited as long as the coating layer (coating film) of the copolymer according to the present invention can be formed. Specifically, a drying temperature is preferably 5 to 50° C. and more preferably 15 to 40° C. A drying step may be performed under a single condition or may be performed stepwise under different conditions. Further, a drying time is preferably 60 to 480 minutes and more preferably 120 to 300 minutes. Further, in a case where the polymer substrate is a porous membrane (hollow fiber membrane), the coating film may be dried by allowing a gas of 5 to 40° C. and more preferably 15 to 30° C. to continuously or gradually circulate on a surface of hollow fiber to which the copolymer-containing solution is applied. Herein, the gas is not particularly limited as long as it has no influence on the coating film (coating layer) and can dry the coating film. Specific examples thereof include air, an inert gas such as nitrogen gas or argon gas, and the like. Further, a circulation amount of the gas is not particularly limited as long as the coating film can be sufficiently dried. The circulation amount of the gas is preferably 5 to 150 L/min and more preferably 30 to 100 L/min.

According to such a method, the copolymer according to the present invention can be efficiently formed on the polymer substrate. Incidentally, depending on the type of cells to be adhered, the polymer substrate may be further treated with a cell adhesion factor such as fibronectin, laminin, or collagen. With such a treatment, adhesion of cells to the substrate surface and growth of cells can be further promoted. In a case where the polymer substrate is a porous membrane formed of a plurality of hollow fibers, the treatment with a cell adhesion factor may be performed with respect to one hollow fiber and then the hollow fibers may be bundled, or a plurality of hollow fibers are bundled to produce a porous membrane and then the treatment may be performed. Further, the treatment with a cell adhesion factor may be performed after the coating layer containing the copolymer according to the present invention is formed, before the coating layer containing the copolymer according to the present invention is formed, or at the same time the coating layer containing the copolymer according to the present invention is formed.

### <Bioreactor>

The cell culture substrate of the present invention shows excellent cell adhesion. Further, the cell culture substrate of the present invention has cell proliferation activity. Therefore, the cell culture substrate of the present invention can be suitably used in a bioreactor. That is, the present invention provides a bioreactor including the cell culture substrate of the present invention. Here, the bioreactor may be a plane type bioreactor or a hollow fiber type bioreactor, but is particularly preferably a hollow fiber type bioreactor. Therefore, in the following description, although a hollow fiber type bioreactor will be described as a preferred embodiment, the bioreactor of the present invention may be a plane type bioreactor, and in this case, the following embodiment can be appropriately changed and applied. Further, dimensional ratios in the drawings are exaggerated for the sake of explanatory convenience and may differ from actual ratios.

The bioreactor in which the cell culture substrate of the present invention can be suitably used is not particularly limited, but the cell culture substrate and the bioreactor of the present invention can be applied, for example, to cell culture/expansion systems described in JP 2010-523118 A (JP 5524824 B2)(WO 2008/124229 A2), JP 2013-524854 A (JP 6039547 B2) (WO 2011/140231 A1), JP 2013-507143 A (JP 5819835 B2) (WO 2011/045644 A1), JP 2013-176377 A (WO 2008/109674), JP 2015-526093 A (WO 2014/031666 A1), JP 2016-537001 A (WO 2015/073918 A1), JP 2017-509344 A (WO 2015/148704 A1), and the like; and Quantum Cell Expansion System manufactured by TERUMO BCT, INC. Conventionally, in the cell culture, facilities such as an incubator, a safety cabinet, and a clean room are separately needed, but the culture system as described above has all of those functions so that the facilities can be very simplified. Further, by controlling temperature or gas during the cell culture using the system as described above, a functionally closed system can be ensured and the cell culture can be performed automatically and in a closed environment.

Hereinafter, an embodiment of the bioreactor of the present invention will be described with reference to the drawings, but the present invention is not limited to the following embodiment.

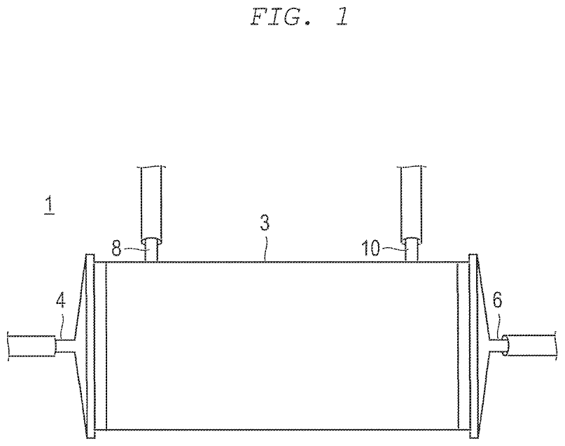

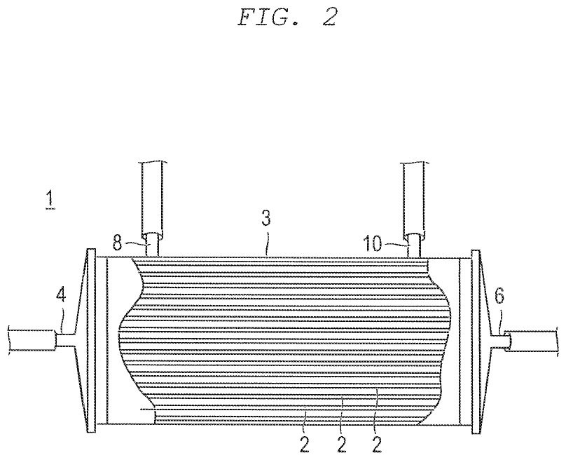

FIG. 1 is a partial side view illustrating an embodiment of a bioreactor (hollow fiber type bioreactor) of the present invention. Further, FIG. 2 is a partially cut-away side view of the bioreactor of FIG. 1. In FIGS. 1 and 2, a bioreactor 1 has a cell culture substrate 2 of the present invention provided in a cell culture chamber 3. The cell culture chamber 3 has four openings, that is, four ports (an inlet port 4, an outlet port 6, an inlet port 8, and an outlet port 10). Herein, a culture medium including cells flows to a hollow fiber intracapillary (IC) space of the cell culture substrate 2 in the cell culture chamber 3 through the inlet port 4, and discharged from the outlet port 6. According to this, cells are efficiently adhered (attached) to and cultured on the surface of the hollow fiber lumen. Meanwhile, a culture medium or gas (such as oxygen or carbon dioxide) flows to be in contact with a hollow fiber extracapillary (EC) space of the cell culture substrate 2 in the cell culture chamber 3 through the inlet port 8, and discharged from the outlet port 10. According to this, in the cell culture chamber 3, small molecules such as culture medium components flow into the hollow fibers or unnecessary components are discharged from the inside of the hollow fibers, and cells adhered onto the surface of the hollow fibers are cultured. Further, after culturing for a predetermined time, a liquid (for example, PBS) containing trypsin is introduced into the intracapillary (IC) space of the hollow fiber of the cell culture substrate 2 in the cell culture chamber 3 through the inlet port 4, and then is held for a predetermined time (for example, about 5 to 10 minutes). Next, a culture medium or an isotonic solution such as PBS flows in the intracapillary (IC) space of the hollow fiber of the cell culture substrate 2 in the cell culture chamber 3 through the inlet port 4 to apply a shear force to cells, the cells are released from the inner wall of the hollow fiber, and the cells are recovered from the bioreactor through the outlet port 6. Incidentally, although the cells are adhered to the intracapillary (IC) space of the hollow fiber in the above embodiment, the present invention is not limited to the above embodiment, and cells may be cultured in such a manner that a culture medium containing cells flows into the outlet port 10 from the inlet port 8, the cells are efficiently adhered (attached) to an outer surface of the hollow fiber, and the culture medium flows into the outlet port 6 from the inlet port 4 in an hollow fiber lumen. Further, the fluid from the inlet port 4 into the outlet port 6 may flow in either a co-current or counter-current direction with respect to flow of fluid into the outlet port 10 from the inlet port 8.

### [Use of Bioreactor]

As mentioned above, the bioreactor of the present invention includes a cell culture substrate excellent in cell adhesion (and further cell proliferation activity). Herein, cells which can be cultured in the bioreactor of the present invention may be adherent (scaffold-dependent) cells, non-adherent cells, or any combination thereof. In consideration of excellent cell adhesion (and further cell proliferation activity), the bioreactor of the present invention can be particularly suitably used in culturing of adherent (scaffold-dependent) cells. Herein, as the adherent (scaffold-dependent) cells, there are animal cells such as stem cells including mesenchymal stem cell (MSC) or the like, fibroblast cells, and the like. As mentioned above, attention has been paid to stem cells in development of regenerative medicine or drug discovery. Therefore, the bioreactor of the present invention can be suitably used in culturing of stem cells. That is, the present invention provides a method for culturing a stem cell using the bioreactor of the present invention. Herein, the method for culturing a stem cell is not particularly limited, and a general culturing method can be applied similarly or appropriately modified.

### EXAMPLES

The effects of the present invention will be described using the following examples and comparative examples. However, the technical scope of the present invention is not limited to only the following examples. Incidentally, in the following examples, operations were carried out at room temperature (25° C.) unless otherwise specified. In addition, unless otherwise specified, “%” and “part” mean “% by weight” and “parts by weight,” respectively.

### Production Example 1: Synthesis of Copolymer (1)

To a 20-ml glass pressure-proof test tube, 1.8 g (0.0138 mol) of methoxyethyl acrylate (MEA), 0.2 g (0.0015 mol) of hydroxyethyl methacrylate (HEMA), and 3 g of methanol were added, and then nitrogen gas was bubbled for 10 seconds, thereby preparing a monomer solution (1). To this monomer solution (1), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (1). The resultant polymerization liquid (1) was added to 50 ml of ethanol, and the precipitated polymer component was recovered and dried under reduced pressure, thereby obtaining a copolymer of methoxyethyl acrylate and hydroxyethyl methacrylate (MEA:HEMA=90:10 (molar ratio)) (copolymer (1)). The weight average molecular weight (Mw) of this copolymer (1) was measured to be 280,000.

### Production Example 2: Synthesis of Copolymer (2)

To a 20-ml glass pressure-proof test tube, 1.6 g (0.0123 mol) of methoxyethyl acrylate (MEA), 0.4 g (0.0031 mol) of hydroxyethyl methacrylate (HEMA), and 3 g of methanol were added, and then nitrogen gas was bubbled for 10 seconds, thereby preparing a monomer solution (2). To this monomer solution (2), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (2). This polymerization liquid (2) was added to 50 ml of ethanol, and the precipitated polymer component was recovered and dried under reduced pressure, thereby obtaining a copolymer of methoxyethyl acrylate and hydroxyethyl methacrylate (MEA:HEMA=80:20 (molar ratio)) (copolymer (2)). The weight average molecular weight (Mw) of this copolymer (2) was measured to be 300,000.

### Production Example 3: Synthesis of Copolymer (3)

To a 20-ml glass pressure-proof test tube, 1.4 g (0.0108 mol) of methoxyethyl acrylate (MEA), 0.6 g (0.0046 mol) of hydroxyethyl methacrylate (HEMA), and 3 g of methanol were added, and then nitrogen gas was bubbled for 10 seconds, thereby preparing a monomer solution (3). To this monomer solution (3), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (3). This polymerization liquid (3) was added to 50 ml of ethanol, and the precipitated polymer component was recovered and dried under reduced pressure, thereby obtaining a copolymer of methoxyethyl acrylate and hydroxyethyl methacrylate (MEA:HEMA=70:30 (molar ratio)) (copolymer (3)). The weight average molecular weight (Mw) of this copolymer (3) was measured to be 290,000.

### Production Example 4: Synthesis of Copolymer (4)

To a 20-ml glass pressure-proof test tube, 1.89 g (0.0145 mol) of methoxyethyl acrylate (MEA), 0.11 g (0.0008 mol) of carboxyethyl acrylate (CEA), and 3 g of methanol were added, and then nitrogen gas was bubbled for 10 seconds, thereby preparing a monomer solution (4). To this monomer solution (4), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (4). This polymerization liquid (4) was added to 50 ml of ethanol, and the precipitated polymer component was recovered and dried under reduced pressure, thereby obtaining a copolymer of methoxyethyl acrylate and carboxyethyl acrylate (MEA:CEA=95:5 (molar ratio)) (copolymer (4)). The weight average molecular weight (Mw) of this copolymer (4) was measured to be 230,000.

### Production Example 5: Synthesis of Copolymer (5)

To a 20-ml glass pressure-proof test tube, 1.78 g (0.0137 mol) of methoxyethyl acrylate (MEA), 0.11 g (0.0015 mol) of carboxyethyl acrylate (CEA), and 3 g of methanol were added, and then nitrogen gas was bubbled for 10 seconds, thereby preparing a monomer solution (5). To this monomer solution (5), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (5). This polymerization liquid (5) was added to 50 ml of ethanol, and the precipitated polymer component was recovered and dried under reduced pressure, thereby obtaining a copolymer of methoxyethyl acrylate and carboxyethyl acrylate (MEA:CEA=90:10 (molar ratio)) (copolymer (5)). The weight average molecular weight (Mw) of this copolymer (5) was measured to be 220,000.

### Production Example 6: Synthesis of Copolymer (6)

To a 20-ml glass pressure-proof test tube, 1.67 g (0.0128 mol) of methoxyethyl acrylate (MEA), 0.33 g (0.0023 mol) of carboxyethyl acrylate (CEA), and 3 g of methanol were added, and then nitrogen gas was bubbled for 10 seconds, thereby preparing a monomer solution (6). To this monomer solution (6), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (6). This polymerization liquid (6) was added to 50 ml of ethanol, and the precipitated polymer component was recovered and dried under reduced pressure, thereby obtaining a copolymer of methoxyethyl acrylate and carboxyethyl acrylate (MEA:CEA=85:15 (molar ratio)) (copolymer (6)). The weight average molecular weight (Mw) of this copolymer (6) was measured to be 190,000.

### Production Example 7: Synthesis of Copolymer (7)

To a 20-ml glass pressure-proof test tube, 1.75 g (0.0134 mol) of methoxyethyl acrylate (MEA), 0.36 g of aqueous solution of 80% trimethylammonium ethyl acrylate (TMAEA) having the following structure (0.288 g, 0.0015 mol as trimethylammonium ethyl acrylate), and

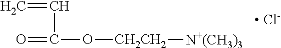

3 g of methanol were added, and then nitrogen gas was bubbled for 10 seconds, thereby preparing a monomer solution (7). To this monomer solution (7), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (7). This polymerization liquid (7) was added to 50 ml of isopropyl alcohol, and the precipitated polymer component was recovered and dried under reduced pressure, thereby obtaining a copolymer of methoxyethyl acrylate and trimethylammonium ethyl acrylate (MEA:TMAEA=90:10 (molar ratio)) (copolymer (7)). The weight average molecular weight (Mw) of this copolymer (7) was measured to be 260,000.

### Production Example 8: Synthesis of Copolymer (8)

To a 20-ml glass pressure-proof test tube, 1.00 g (0.0077 mol) of methoxyethyl acrylate (MEA), 1.24 g of aqueous solution of 80% trimethylammonium ethyl acrylate (TMAEA) having the following structure (0.99 g, 0.0051 mol as trimethylammonium ethyl acrylate), and

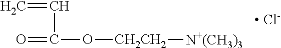

3 g of methanol were added, and then nitrogen gas was bubbled for 10 seconds, thereby preparing a monomer solution (8). To this monomer solution (8), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (8). This polymerization liquid (8) was added to 50 ml of isopropyl alcohol, and the precipitated polymer component was recovered and dried under reduced pressure, thereby obtaining a copolymer of methoxyethyl acrylate and trimethylammonium ethyl acrylate (MEA:TMAEA=60:40 (molar ratio)) (copolymer (8)). The weight average molecular weight (Mw) of this copolymer (8) was measured to be 210,000.

### Production Example 9: Synthesis of Copolymer (9)

To a 20-ml glass pressure-proof test tube, 0.60 g (0.0046 mol) of methoxyethyl acrylate (MEA), 1.70 g of aqueous solution of 80% trimethylammonium ethyl acrylate (TMAEA) having the following structure (1.36 g, 0.0070 mol as trimethylammonium ethyl acrylate), and

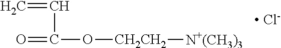

3 g of methanol were added, and then nitrogen gas was bubbled for 10 seconds, thereby preparing a monomer solution (9). To this monomer solution (9), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (9). This polymerization liquid (9) was added to 50 ml of isopropyl alcohol, and the precipitated polymer component was recovered and dried under reduced pressure, thereby obtaining a copolymer of methoxyethyl acrylate and trimethylammonium ethyl acrylate (MEA:TMAEA=40:60 (molar ratio)) (copolymer (9)). The weight average molecular weight (Mw) of this copolymer (9) was measured to be 190,000.

### Production Example 10: Synthesis of MEA Polymer (10)

To a 20-ml glass pressure-proof test tube, 2.0 g (0.0154 mol) of methoxyethyl acrylate (MEA) and 3 g of methanol were added, and then nitrogen gas was bubbled for 10 seconds, thereby preparing a monomer solution (10). To this monomer solution (10), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (10). This polymerization liquid (10) was added to 50 ml of ethanol, and the precipitated polymer component was recovered and dried under reduced pressure, thereby obtaining a homopolymer of methoxyethyl acrylate (MEA polymer (10)). The weight average molecular weight (Mw) of this MEA polymer (10) was measured to be 380,000.

### Production Example 11: Synthesis of Copolymer (11)

To a 20-ml glass pressure-proof test tube, 1.2 g (0.0092 mol) of methoxyethyl acrylate (MEA), 0.8 g (0.0061 mol) of hydroxyethyl methacrylate (HEMA), and 3 g of methanol were added, and then nitrogen gas was bubbled for 10 seconds, thereby preparing a monomer solution (11). To this monomer solution (11), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (11). This polymerization liquid (11) was added to 50 ml of ethanol, and the precipitated polymer component was recovered and dried under reduced pressure, thereby obtaining a copolymer of methoxyethyl acrylate and hydroxyethyl methacrylate (MEA:HEMA=60:40 (molar ratio)) (copolymer (11)). The weight average molecular weight (Mw) of this copolymer (11) was measured to be 280,000.

### Production Example 12: Synthesis of Copolymer (12)

To a 20-ml glass pressure-proof test tube, 1.60 g (0.0123 mol) of methoxyethyl acrylate (MEA), 0.44 g (0.0031 mol) of carboxyethyl acrylate (CEA), and 3 g of methanol were added, and then nitrogen gas was bubbled for 10 seconds, thereby preparing a monomer solution (12). To this monomer solution (12), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (12). This polymerization liquid (12) was added to 50 ml of ethanol, and the precipitated polymer component was recovered and dried under reduced pressure, thereby obtaining a copolymer of methoxyethyl acrylate and carboxyethyl acrylate (MEA:CEA=80:20 (molar ratio)) (copolymer (12)). The weight average molecular weight (Mw) of this copolymer (12) was measured to be 185,000.

### Production Example 13: Synthesis of Copolymer (13)

To a 20-ml glass pressure-proof test tube, 0.30 g (0.0023 mol) of methoxyethyl acrylate (MEA), 2.20 g of aqueous solution of 80% trimethylammonium ethyl acrylate (TMAEA) having the following structure (1.76 g, 0.0091 mol as trimethylammonium ethyl acrylate), and

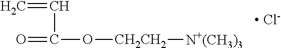

3 g of methanol were added, and then nitrogen gas was bubbled for 10 seconds, thereby preparing a monomer solution (13). To this monomer solution (13), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining a polymerization liquid (13). This polymerization liquid (13) was added to 50 ml of isopropyl alcohol, and the precipitated polymer component was recovered and dried under reduced pressure, thereby obtaining a copolymer of methoxyethyl acrylate and trimethylammonium ethyl acrylate (MEA:TMAEA=20:80 (molar ratio)) (copolymer (13)). The weight average molecular weight (Mw) of this copolymer (13) was measured to be 150,000.

### Example 1: Coating to Cell Culture Dish

The copolymer (1) obtained in Production Example 1 was dissolved in methanol to have a concentration of 0.005% by weight, thereby preparing a coating liquid (1). 25 μL of the coating liquid (1) was added to each well of commercially available 96-well tissue culture polystyrene dish (without a plasma treatment, manufactured by FALCON, trade name: Non-Tissue Culture Treated Plate, 96 Well, Flat Bottom with Low Evaporation Lid), and dried at 20° C. for 300 minutes to produce a polymer coating film (dry thickness: 33 nm), thereby obtaining a cell culture dish (1).

### Examples 2 to 9: Coating to Cell Culture Dish

A polymer coating film was produced on a well surface according to the similar method to Example 1, except that, in Example 1, each of the copolymers (2) to (9) was used instead of the copolymer (1), thereby obtaining cell culture dishes (2) to (9).

### Comparative Examples 1 to 5: Coating to Cell Culture Dish

A polymer coating film was produced on a well surface according to the similar method to Example 1, except that, in Example 1, each of the MEA polymer (10) and the copolymers (11) to (13) was used instead of the copolymer (1), thereby obtaining comparative cell culture dishes (1) to (4). Further, a commercially available 96-well tissue culture polystyrene dish (without a plasma treatment and a polymer coating film, manufactured by FALCON, trade name: Non-Tissue Culture Treated Plate, 96 Well, Flat Bottom with Low Evaporation Lid) was used as a comparative cell culture dish (5).

### Reference Example 1: Plasma Treated Cell Culture Dish

A commercially available 96-well tissue culture polystyrene dish (with a plasma treatment, manufactured by FALCON, trade name: Tissue Culture Treated Plate, 96. Well, Flat Bottom with Low Evaporation Lid) was used as a reference cell culture dish (1).

### Examples 10 to 18, Comparative Examples 6 to 10, and Reference Example 2: Cell Culture and Measurement of Adhesion Activity

By the following method, cells were cultured using the cell culture dishes (1) to (9) and the comparative cell culture dishes (1) to (5) obtained in Examples 1 to 9 and Comparative Examples 1 to 5, and the plasma treated cell culture dish obtained in Reference Example 1, and the cell adhesion activity (cell adhesion) was evaluated. Incidentally, as the cells, human adipose tissue-derived mesenchymal stem cells (Lonza, Walkersville, Md., U.S.A.) were used. The donor was a 22-year-old man and expressed CD13, CD29, CD44, CD73, CD90, CD105, CD166≥90%, CD14, CD31, and CD45≤5%.

The human adipose tissue-derived mesenchymal stem cells were seeded on each well of the cell culture dishes (1) to (9), the comparative cell culture dishes (1) to (5), and the plasma treated cell culture dish to be 2×10³ cells/well, and then cultured for one day in Mesenchymal Stem Cell Growth Medium 2 (PromoCell GmbH, Bedford, Mass., U.S.A.) in an incubator under humidified conditions at 37° C. in the presence of 5% CO₂. After the completion of culture, the culture solution was exchanged with Mesenchymal Stem Cell Growth Medium 2 containing 10% WST-1 (Premix WST-1 Cell Proliferation Assay System, Takara Bio Inc., Shiga, Japan) and then incubated for about 4 hours under normal pressure (37° C., 5% CO₂) under humidified conditions. An absorbance (450 nm, comparison 600 nm) of the culture solution was measured by a microplate reader and regarded as cell adhesion activity. Results are presented in the following Table 1. In the following Table 1, a proportion of absorbance of the culture solution cultured in each culture dish in a case where the absorbance the culture solution cultured in the comparative cell culture dish (1) (Comparative Example 6) is regarded as “1.00” is shown as a proportion of adhesion activity.

TABLE 1

| | Polymer | | | | Ratio of adhesion |
|---|---|---|---|---|---|
| | | | Monomer | Cell adhesion | activity (to |
| | | | composition | activity | Comparative |
| | | Monomer type | (molar ratio) | (Abs₄₅₀) | Example 6) |
| Example 10 | Copolymer (1) | MEA-HEMA | 90:10 | 0.066 | 1.10 |
| Example 11 | Copolymer (2) | MEA-HEMA | 80:20 | 0.064 | 1.07 |
| Example 12 | Copolymer (3) | MEA-HEMA | 70:30 | 0.063 | 1.05 |
| Example 13 | Copolymer (4) | MEA-CEA | 95:5 | 0.067 | 1.12 |
| Example 14 | Copolymer (5) | MEA-CEA | 90:10 | 0.066 | 1.10 |
| Example 15 | Copolymer (6) | MEA-CEA | 85:15 | 0.064 | 1.07 |
| Example 16 | Copolymer (7) | MEA-TMAEA | 90:10 | 0.061 | 1.02 |
| Example 17 | Copolymer (8) | MEA-TMAEA | 60:40 | 0.064 | 1.07 |
| Example 18 | Copolymer (9) | MEA-TMAEA | 40:60 | 0.061 | 1.02 |
| Comparative | MEA polymer (10) | MEA | 100:0 | 0.060 | 1.00 |
| Example 6 | | | | | |
| Comparative | Copolymer (11) | MEA-HEMA | 60:40 | 0.058 | 0.97 |
| Example 7 | | | | | |
| Comparative | Copolymer (12) | MEA-CEA | 80:20 | 0.060 | 1.00 |
| Example 8 | | | | | |
| Comparative | Copolymer (13) | MEA-TMAEA | 20:80 | 0.057 | 0.95 |
| Example 9 | | | | | |

| | | | |
|---|---|---|---|
| Comparative | - (Without plasma treatment) | 0.047 | 0.78 |
| Example 10 | | | |
| Reference | - (With plasma treatment) | 0.066 | 1.10 |
| Example 2 | | | |

It is noted from the results of Table 1 that the cell culture dishes (1) to (9) having the polymer coating films of the copolymers (1) to (9) of Production Examples 1 to 9 formed thereon show excellent cell adhesion, as compared to the those of MEA polymer (10) and MEA-HEMA copolymer, MEA-CEA copolymer, and MEA-TMAEA copolymer which are out of the composition according to the present invention.

### REFERENCE SIGNS LIST

1 BIOREACTOR
2 CELL CULTURE SUBSTRATE
3 CELL CULTURE CHAMBER
4, 8 INLET PORT
6, 10 OUTLET PORT

1. A cell culture substrate comprising a coating layer on at least one surface of a polymer substrate, wherein the coating layer includes at least one member selected from the group consisting of
(a) a copolymer (a) comprising 40% by mole or more and 90% by mole or less of a structural unit (a-1) derived from alkoxyalkyl (meth)acrylate of following Formula (1) and 10% by mole or more and 60% by mole or less of a structural unit (a-2) derived from trialkyl aminoalkyl (meth)acrylate of following Formula (2) (a total of the structural unit (a-1) and the structural unit (a-2) is 100% by mole),
(b) a copolymer (b) comprising 85% by mole or more and 95% by mole or less of a structural unit (b-1) derived from alkoxyalkyl (meth)acrylate of following Formula (1) and 5% by mole or more and 15% by mole or less of a structural unit (b-2) derived from carboxyalkyl (meth)acrylate of following Formula (3) (a total of the structural unit (b-1) and the structural unit (b-2) is 100% by mole), and
(c) a copolymer (c) comprising 70% by mole or more and 90% by mole or less of a structural unit (c-1) derived from alkoxyalkyl (meth)acrylate of following Formula (1) and 10% by mole or more and 30% by mole or less of a structural unit (c-2) derived from hydroxyalkyl (meth)acrylate of following Formula (4) (a total of the structural unit (c-1) and the structural unit (c-2) is 100% by mole): 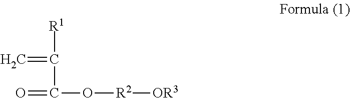
wherein R¹ represents a hydrogen atom or a methyl group, R² represents an alkylene group having 2 or 3 carbon atoms, and R³ represents an alkyl group having 1 to 3 carbon atoms, 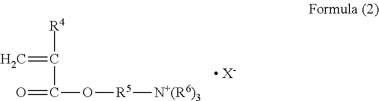
wherein R⁴ represents a hydrogen atom or a methyl group, R⁵ represents an alkylene group having 2 or 3 carbon atoms, R⁶ each independently represents an alkyl group having 1 to 3 carbon atoms, and X⁻ represents an anion moiety, 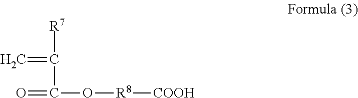
wherein R⁷ represents a hydrogen atom or a methyl group, and R⁸ represents an alkylene group having 2 or 3 carbon atoms, Formula (4): 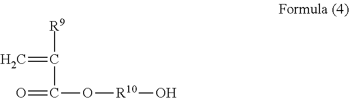
wherein R⁹ represents a hydrogen atom or a methyl group, and R¹⁰ represents an alkylene group having 2 or 3 carbon atoms.

2. The cell culture substrate according to claim 1, wherein the copolymer (a) is a copolymer comprising 50% by mole or more and 70% by mole or less of the structural unit (a-1) derived from alkoxyalkyl (meth)acrylate of the Formula (1) and 30% by mole or more and 50% by mole or less of the structural unit (a-2) derived from trialkyl aminoalkyl (meth)acrylate of the Formula (2) (the total of the structural unit (a-1) and the structural unit (a-2) is 100% by mole).

3. The cell culture substrate according to claim 1, wherein the copolymer (a) is composed of the structural unit (a-1) and the structural unit (a-2).

4. The cell culture substrate according to claim 1, wherein the copolymer (b) is a copolymer comprising 90% by mole or more and 95% by mole or less of the structural unit (b-1) derived from alkoxyalkyl (meth)acrylate of the Formula (1) and 5% by mole or more and 10% by mole or less of the structural unit (b-2) derived from carboxyalkyl (meth)acrylate of the Formula (3) (the total of the structural unit (b-1) and the structural unit (b-2) is 100% by mole).

5. The cell culture substrate according to claim 1, wherein the copolymer (b) is composed of the structural unit (b-1) and the structural unit (b-2).

6. The cell culture substrate according to claim 1, wherein the copolymer (c) is a copolymer comprising 80% by mole or more and 90% by mole or less of the structural unit (c-1) derived from alkoxyalkyl (meth)acrylate of the Formula (1) and 10% by mole or more and 20% by mole or less of the structural unit (c-2) derived from hydroxyalkyl (meth)acrylate of the Formula (4) (the total of the structural unit (c-1) and the structural unit (c-2) is 100% by mole).

7. The cell culture substrate according to claim 1, wherein the copolymer (c) is composed of the structural unit (c-1) and the structural unit (c-2).

8. The cell culture substrate according to claim 1, wherein the polymer substrate is a porous membrane.

9. A bioreactor comprising the cell culture substrate according to claim 1.

10. A method for culturing a stem cell using the bioreactor according to claim 9.
